(19) United States
(12) Patent Application Publication
(10) Pub. No.: US 2019/0183972 A1
(43) Pub. Date: Jun. 20, 2019

(54) COMBINATION OF RAMUCIRUMAB AND PEMBROLIZUMAB FOR THE TREATMENT OF CERTAIN CANCERS

(71) Applicants: ImClone LLC, Indianapolis, IN (US); Merck Sharp & Dohme Corp, Rahway, NJ (US)

(72) Inventor: Richard Brian GAYNOR, Carmel, IN (US)

(21) Appl. No.: 16/305,327

(22) PCT Filed: May 26, 2017
(86) PCT No.: PCT/US2017/034732

§ 371 Date: Nov. 28, 2018

Related U.S. Application Data
(60) Provisional application No. 62/469,670, filed on Mar. 10, 2017.
(60) Provisional application No. 62/460,404, filed on Feb. 17, 2017.
(60) Provisional application No. 62/434,466, filed on Dec. 15, 2016.
(60) Provisional application No. 62/398,663, filed on Sep. 23, 2016.
(60) Provisional application No. 62/377,852, filed on Aug. 22, 2016.
(60) Provisional application No. 62/345,322, filed on Jun. 3, 2016.

(51) Int. Cl. A61K 38/17; A61P 35/00

(52) U.S. Cl. CPC A61K 38/1774; A61P 35/00

(57) ABSTRACT

The present disclosure relates to a combination of ramucirumab and pembrolizumab and methods of using the combination to treat certain disorders, such as non-small cell lung cancer, urothelial cancer, biliary tract cancer, and advanced gastric or gastroesophageal junction adenocarcinoma.

The present invention relates to a combination of ramucirumab and pembrolizumab, and to methods of using the combination to treat certain disorders, such as non-small cell lung cancer (NSCLC), urothelial cancer, biliary tract cancer, and advanced gastric or gastroesophageal junction (G/GEJ) adenocarcinoma.

Hallmarks of tumor growth include angiogenesis and immunosuppression. Programmed death receptor-1 (programed death-1 or PD-1) is expressed on the cell surface of activated T-cells under healthy conditions. The normal function of PD-1 is to down-modulate unwanted or excessive immune responses, including autoimmune reactions. Programmed death ligand-1 (PD-L1) is a ligand to PD-1, and suppresses T-cell migration, proliferation and secretion of cytotoxic mediators, and restricts tumor cell killing. Herbst et al. Nature 2014; 515:63-567. The PD-1/PD-L1 interaction is a major pathway hijacked by tumors to suppress immune control.

Simultaneously targeting both angiogenesis and immunosuppression by an anti-angiogenic antibody and a PD-1 antibody has shown synergistic effects in preclinical studies. Yasuda S, et al. Clin Exp Immunol. 2013; 172:500-6. Blocking vascular endothelial growth factor A (VEGF-A) and vascular endothelial growth factor receptor-2 (VEGFR-2) can relieve T cell exhaustion by reverting the expression of inhibitory molecules, including PD-1. Voron T, et al. J. Exp. Med. 2015; 212:139-48. Anti-VEGFR-2 antibodies have been shown to improve T cell infiltration into tumors and inhibit migration of tumor associated macrophages in preclinical studies. Manning E A, et al. Clin Cancer Res. 2007; 13:3951-9; Dineen S P, et al. Cancer Res. 2008; 68:4340-6. Work has been done to elucidate a correlation between tumors expressing high levels of PD-L1—especially PD-L1 expressed on tumor-infiltrating immune cells—and response to treatment with anti-PD-L1 antibodies. Herbst et al. Nature 2014; 515:63-567. In pembrolizumab trials, response rate has been shown to increase approximately 2-fold in PD-L1 strong positive patients in NSCLC and head and neck cancer (Chow et al., J. Clin. Oncol. 34(32):3838-3847 (2016); Garon et al., N. Engl. J. Med. 372(21):2018-28 (2015).

Ramucirumab (a non-limiting example of which is CYRAMZA®, Eli Lilly & Co., Indianapolis, Ind.) is a human IgG1 monoclonal antibody directed against the vascular endothelial growth factor receptor 2 (VEGFR-2). Ramucirumab and methods of making and using this compound are disclosed in WO2003/075840. Ramucirumab is approved by the United States Food and Drug Administration as a single agent or in combination with paclitaxel, for the treatment of advanced gastric or gastroesophageal junction adenocarcinoma, with disease progression on or after prior fluoropyrimidine- or platinum-containing chemotherapy; in combination with docetaxel, for the treatment of metastatic non-small cell lung cancer with disease progression on or after platinum-based chemotherapy. Patients with EGFR or ALK genomic tumor aberrations should have disease progression on FDA-approved therapy for these aberrations prior to receiving CYRAMZA®; and in combination with FOLFIRI (irinotecan, folinic acid, and 5-fluorouracil), for the treatment of metastatic colorectal cancer with disease progression on or after prior therapy with bevacizumab, oxaliplatin, and a fluoropyrimidine.

Pembrolizumab (a non-limiting example of which is KEYTRUDA®, Merck & Co., Inc., Whitehouse Station, N.J., USA) is a humanized IgG4 monoclonal antibody against programmed death receptor-1 (PD-1). Pembrolizumab and methods of making and using this compound are disclosed in WO2008156712. Pembrolizumab has been shown to inhibit the binding of PD-1 to PD-L1 and PD-L2, and has been tested in various clinical trials. (WO2008156712 and Hamid et al., N. Engl. J. Med. (2013) 369:2). Pembrolizumab is approved by the US Food and Drug Administration (FDA) for the treatment of patients with unresectable or metastatic melanoma, patients with metastatic NSCLC whose tumors have high PD-L1 expression as determined by an FDA-approved test with no EGFR or ALK genomic tumor aberrations, and no prior systemic chemotherapy treatment, and for patients with metastatic NSCLC whose tumors express PD-L1 and who have disease progression on or after platinum-containing chemotherapy. Patients with EGFR or ALK genomic tumor aberrations should have disease progression on FDA-approved therapy for these aberrations prior to receiving pembrolizumab. Pembrolizumab is also approved for patients with recurrent or metastatic head and neck squamous cell carcinoma (HNSCC) with disease progression on or after platinum-containing chemotherapy and for adult and pediatric patients with refractory classical Hodgkin lymphoma or who have relapsed after 3 or more prior lines of therapy.

The present invention is derived from the ongoing Phase I clinical trial of the combination of ramucirumab and pembrolizumab (“A Phase 1 Study of ramucirumab plus pembrolizumab in patients with advanced gastric or gastroesophageal junction (G/GEJ) adenocarcinoma, non-small cell lung cancer (NSCLC), or urothelial carcinoma (UC)” (the “Study”).

Although combinations of inhibitors of VEGFR-2 and PD-1 have been contemplated in the art, surprisingly, the present invention discloses the combination of ramucirumab and pembrolizumab as part of an effective treatment regimen in second to fourth line NSCLC patients, as demonstrated by 85.0% of patients experiencing a decrease in target lesions, and as part of an effective treatment regimen in advanced gastric or gastroesophageal junction (G/GEJ) adenocarcinoma patients as demonstrated by 45% of patients experiencing a decrease in target lesions. Based on this, ongoing protocol amendments include new cohorts for 1st line NSCLC, 1st line gastric/gastroesophageal junction, and 2nd-3rd line biliary tract cancer.

Also surprising is that the combination of the present invention tends to show effects in patients with both PD-L1 positive and PD-L1 negative status in non-small cell lung cancer.

Given the importance of angiogenesis and immunosuppression in tumor growth, there exists a need for combination therapies to improve responses in specific cancers.

According to the first aspect of the present invention, there is presented a method of treating non-small cell lung cancer in a patient, comprising administering to a non-small cell lung cancer patient in need of such treatment an effective amount of an anti-VEGFR-2 antibody comprising two light chains, each having the amino acid sequence of SEQ ID NO: 3 and two heavy chains, each having the amino acid sequence of SEQ ID NO: 4, and an effective amount of an anti-PD-1 antibody comprising two light chains, each having the amino acid sequence of SEQ ID NO: 5 and two heavy chains, each having the amino acid sequence of SEQ ID NO: 6.

Another aspect of the invention is a method of treating advanced gastric or gastroesophageal junction adenocarcinoma in a patient, comprising administering to an advanced gastric or gastroesophageal junction adenocarcinoma patient in need of such treatment an effective amount of an anti-VEGFR-2 antibody comprising two light chains, each having the amino acid sequence of SEQ ID NO: 3 and two heavy chains, each having the amino acid sequences of SEQ ID NO: 4, and an effective amount of an anti-PD-1 antibody comprising two light chains, each having the amino acid sequence of SEQ ID NO: 5 and two heavy chains, each having the amino acid sequence of SEQ ID NO: 6.

Another aspect of the invention is a kit comprising an anti-VEGFR-2 antibody comprising two light chains, each having the amino acid sequence of SEQ ID NO: 3 and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6 for the treatment of non-small cell lung cancer.

Another aspect of the invention is a kit comprising an anti-VEGFR-2 antibody comprising two light chains, each having the amino acid sequence of SEQ ID NO: 3, and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6 for the treatment of advanced gastric or gastroesophageal junction adenocarcinoma.

In a preferred aspect of the invention the anti-VEGFR-2 antibody is ramucirumab and the anti-PD-1 antibody is pembrolizumab.

In another preferred aspect of the invention the anti-VEGFR-2 antibody comprises a light chain variable region having the amino acid sequence of SEQ ID NO:1 and a heavy chain variable region having the amino acid sequence of SEQ ID NO:2.

In another preferred aspect of the invention the an anti-PD-1 antibody comprises a light chain variable region having the amino acid sequence of SEQ ID NO: 7 and a heavy chain variable region having the amino acid sequence of SEQ ID NO: 8.

In another preferred aspect of the invention the anti-VEGFR-2 antibody comprises a light chain having the amino acid sequence of SEQ ID NO:3 and a heavy chain having the amino acid sequence of SEQ ID NO:4.

In another preferred aspect of the invention ramucirumab is administered once every three weeks at 10 mg/kg and pembrolizumab is administered once every three weeks at 200 mg.

Another aspect of the invention is a kit comprising ramucirumab with one or more pharmaceutically acceptable carriers, diluents, or excipients, and pembrolizumab with one or more pharmaceutically acceptable carriers, diluents, or excipients, for the treatment of non-small cell lung cancer.

Another aspect of the invention is a kit comprising ramucirumab with one or more pharmaceutically acceptable carriers, diluents, or excipients, and pembrolizumab with one or more pharmaceutically acceptable carriers, diluents, or excipients, for the treatment of advanced gastric or gastroesophageal junction adenocarcinoma.

Another aspect of the invention is a combination comprising ramucirumab and pembrolizumab for simultaneous, separate or sequential use in the treatment of non-small cell lung cancer.

Another aspect of the invention is a combination comprising ramucirumab and pembrolizumab for simultaneous, separate or sequential use in the treatment of advanced gastric or gastroesophageal junction adenocarcinoma.

Another aspect of the invention is ramucirumab for use in simultaneous, separate or sequential treatment with pembrolizumab in the treatment of non-small cell lung cancer.

Another aspect of the invention is ramucirumab for use in simultaneous, separate or sequential treatment with pembrolizumab in the treatment of advanced gastric or gastroesophageal junction adenocarcinoma.

Another aspect of the invention is pembrolizumab and ramucirumab for use in the treatment of a non-small cell lung tumor in a patient, wherein the patient has PD-L1 negative or positive status.

Another aspect of the invention is pembrolizumab and ramucirumab for use in the treatment of an advanced gastric or gastroesophageal junction adenocarcinoma in a patient, wherein the patient has PD-L1 negative or positive status.

Another aspect of the invention is the use of ramucirumab and pembrolizumab in the manufacture of a medicament for the treatment of a patient with a non-small cell lung tumor.

Another aspect of the invention is the use of ramucirumab and pembrolizumab in the manufacture of a medicament for the treatment of a patient with advanced gastric or gastroesophageal junction adenocarcinoma.

In a preferred aspect of the invention the patient has PD-L1 negative status.

Another aspect of the invention is a method of treating non-small cell lung cancer in a patient, comprising administering an effective amount of ramucirumab in combination with pembrolizumab to the patient in need thereof, provided that the patient is selected for treatment if the patient has PD-L1 negative or positive status.

Another aspect of the invention is a method of treating advanced gastric or gastroesophageal junction adenocarcinoma in a patient, comprising administering an effective amount of ramucirumab in combination with pembrolizumab to the patient in need thereof, provided that the patient is selected for treatment if the patient has PD-L1 negative or positive status.

Another aspect of the invention is a method of treating non-small cell lung cancer in a patient, comprising testing the patient for the presence of PD-L1 prior to administering ramucirumab in combination with pembrolizumab, and administering to the patient an effective amount of ramucirumab in combination with pembrolizumab if the patient has positive or negative PD-L1 status.

Another aspect of the invention is a method of treating advanced gastric or gastroesophageal junction adenocarcinoma in a patient, comprising testing the patient for the presence of PD-L1 prior to administering ramucirumab in combination with pembrolizumab, and administering to the patient an effective amount of ramucirumab in combination with pembrolizumab if the patient has positive or negative PD-L1 status.

Another aspect of the invention is a method of treating non-small cell lung cancer, locally advanced and unresectable or metastatic gastric or gastroesophageal junction adenocarcinoma, urothelial cancer, or biliary tract cancer in a patient, comprising administering to the patient in need of such treatment an effective amount of an anti-VEGFR-2 antibody comprising two light chains, each having the amino acid sequence of SEQ ID NO: 3 and two heavy chains, each having the amino acid sequence of SEQ ID NO: 4, and an effective amount of an anti-PD-1 antibody comprising two light chains, each having the amino acid sequence of SEQ ID NO: 5 and two heavy chains, each having the amino acid sequence of SEQ ID NO: 6; wherein the anti-PD-1 antibody is administered at a dose of 200 mg, once every three weeks.

Another aspect of the invention is a method of treating locally advanced and unresectable or metastatic gastric or gastroesophageal junction adenocarcinoma or biliary tract cancer in a patient, comprising administering to the patient in need of such treatment an effective amount of an anti-VEGFR-2 antibody comprising two light chains, each having the amino acid sequence of SEQ ID NO: 3 and two heavy chains, each having the amino acid sequence of SEQ ID NO: 4, and an effective amount of an anti-PD-1 antibody comprising two light chains, each having the amino acid sequence of SEQ ID NO: 5 and two heavy chains, each having the amino acid sequence of SEQ NO: 6; wherein the anti-PD-1 antibody is administered at a dose of 200 mg, once every three weeks; wherein the anti-VEGFR-2 antibody is administered at a dose of 8 mg/kg on Day 1 and Day 8 of a three week cycle.

Another aspect of the invention is a method of treating locally advanced and unresectable or metastatic gastric or gastroesophageal junction adenocarcinoma in a patient, comprising administering to the patient in need of such treatment an effective amount of an anti-VEGFR-2 antibody comprising two light chains, each having the amino acid sequence of SEQ ID NO: 3 and two heavy chains, each having the amino acid sequence of SEQ ID NO: 4, and an effective amount of an anti-PD-1 antibody comprising two light chains, each having the amino acid sequence of SEQ ID NO: 5 and two heavy chains, each having the amino acid sequence of SEQ ID NO: 6; wherein the anti-PD-1 antibody is administered at a dose of 200 mg, once every three weeks; wherein the anti-VEGFR-2 antibody is administered at a dose of 8 mg/kg on Day 1 and Day 8 of a three week cycle.

Another aspect of the invention is a method of treating biliary tract cancer in a patient, comprising administering to the patient in need of such treatment an effective amount of an anti-VEGFR-2 antibody comprising two light chains, each having the amino acid sequence of SEQ ID NO: 3 and two heavy chains, each having the amino acid sequence of SEQ ID NO: 4, and an effective amount of an anti-PD-1 antibody comprising two light chains, each having the amino acid sequence of SEQ ID NO: 5 and two heavy chains, each having the amino acid sequence of SEQ ID NO: 6; wherein the anti-PD-1 antibody is administered at a dose of 200 mg, once every three weeks; wherein the anti-VEGFR-2 antibody is administered at a dose of 8 mg/kg on Day 1 and Day 8 of a three week cycle.

Another aspect of the invention is a method of treating non-small cell lung cancer, locally advanced and unresectable or metastatic gastric or gastroesophageal junction adenocarcinoma, or urothelial cancer in a patient, comprising administering to the patient in need of such treatment an effective amount of an anti-VEGFR-2 antibody comprising two light chains, each having the amino acid sequence of SEQ ID NO: 3 and two heavy chains, each having the amino acid sequence of SEQ ID NO: 4, and an effective amount of an anti-PD-1 antibody comprising two light chains, each having the amino acid sequence of SEQ ID NO: 5 and two heavy chains, each having the amino acid sequence of SEQ ID NO: 6; wherein the anti-PD-1 antibody is administered at a dose of 200 mg, once every three weeks; wherein the anti-VEGFR-2 antibody is administered at a dose of 10 mg/kg once every three weeks.

Another aspect of the invention is a method of treating non-small cell lung cancer in a patient, comprising administering to the patient in need of such treatment an effective amount of an anti-VEGFR-2 antibody comprising two light chains, each having the amino acid sequence of SEQ ID NO: 3 and two heavy chains, each having the amino acid sequence of SEQ ID NO: 4, and an effective amount of an anti-PD-1 antibody comprising two light chains, each having the amino acid sequence of SEQ ID NO: 5 and two heavy chains, each having the amino acid sequence of SEQ ID NO: 6; wherein the anti-PD-1 antibody is administered at a dose of 200 mg, once every three weeks; wherein the anti-VEGFR-2 antibody is administered at a dose of 10 mg/kg once every three weeks.

Another aspect of the invention is a method of treating locally advanced and unresectable or metastatic gastric or gastroesophageal junction adenocarcinoma in a patient, comprising administering to the patient in need of such treatment an effective amount of an anti-VEGFR-2 antibody comprising two light chains, each having the amino acid sequence of SEQ ID NO: 3 and two heavy chains, each having the amino acid sequence of SEQ ID NO: 4, and an effective amount of an anti-PD-1 antibody comprising two light chains, each having the amino acid sequence of SEQ ID NO: 5 and two heavy chains, each having the amino acid sequence of SEQ ID NO: 6; wherein the anti-PD-1 antibody is administered at a dose of 200 mg, once every three weeks; wherein the anti-VEGFR-2 antibody is administered at a dose of 10 mg/kg once every three weeks.

Another aspect of the invention is a method of treating urothelial cancer in a patient, comprising administering to the patient in need of such treatment an effective amount of an anti-VEGFR-2 antibody comprising two light chains, each having the amino acid sequence of SEQ ID NO: 3 and two heavy chains, each having the amino acid sequence of SEQ ID NO: 4, and an effective amount of an anti-PD-1 antibody comprising two light chains, each having the amino acid sequence of SEQ ID NO: 5 and two heavy chains, each having the amino acid sequence of SEQ ID NO: 6; wherein the anti-PD-1 antibody is administered at a dose of 200 mg, once every three weeks; wherein the anti-VEGFR-2 antibody is administered at a dose of 10 mg/kg once every three weeks.

Another aspect of the invention is an anti-VEGFR-2 antibody comprising two light chains, each having the amino acid sequence of SEQ ID NO: :3 and two heavy chains, each having the amino acid sequence of SEQ ID NO: 4, for use in simultaneous, separate, or sequential combination with an anti-PD-1 antibody comprising two light chains, each having the amino acid sequence of SEQ ID NO: 5 and two heavy chains, each having the amino acid sequence of SEQ ID NO: 6, in the treatment of non-small cell lung cancer, locally advanced and unresectable or metastatic gastric or gastroesophageal junction adenocarcinoma, urothelial cancer, or binary tract cancer; wherein the anti-PD-1 antibody is administered at a dose of 200 mg, once every three weeks.

Another aspect of the invention is an anti-VEGFR-2 antibody comprising two light chains, each having the amino acid sequence of SEQ ID NO: 3 and two heavy chains, each having the amino acid sequence of SEQ ID NO: 4, for use in simultaneous, separate, or sequential combination with an anti-PD-1 antibody comprising two light chains, each having the amino acid sequence of SEQ ID NO: 5 and two heavy chains, each having the amino acid sequence of SEQ ID NO: 6, in the treatment of locally advanced and unresectable or metastatic gastric or gastroesophageal junction adenocarcinoma or biliary tract cancer; wherein the anti-PD-1 antibody is administered at a dose of 200 mg, once every three weeks; wherein the anti-VEGFR-2 antibody is administered at a dose of 8 mg/kg on Day 1 and Day 8 of a three week cycle.

Another aspect of the invention is an anti-VEGFR-2 antibody comprising two light chains, each having the amino acid sequence of SEQ ID NO: 3 and two heavy chains, each having the amino acid sequence of SEQ ID NO: 4, for use in simultaneous, separate, or sequential combination with an anti-PD-1 antibody comprising two light chains, each having the amino acid sequence of SEQ ID NO: 5 and two heavy chains, each having the amino acid sequence of SEQ ID NO: 6, in the treatment of locally advanced and unresectable or metastatic gastric or gastroesophageal junction adenocarcinoma; wherein the anti-PD-1 antibody is administered at a dose of 200 mg, once every three weeks; wherein the anti-VEGFR-2 antibody is administered at a dose of 8 mg/kg on Day 1 and Day 8 of a three week cycle.

Another aspect of the invention is an anti-VEGFR-2 antibody comprising two light chains, each having the amino acid sequence of SEQ ID NO: 3 and two heavy chains, each having the amino acid sequence of SEQ ID NO: 4, for use in simultaneous, separate, or sequential combination with an anti-PD-1 antibody comprising two light chains, each having the amino acid sequence of SEQ ID NO: 5 and two heavy chains, each having the amino acid sequence of SEQ ID NO: 6, in the treatment of biliary tract cancer; wherein the anti-PD-1 antibody is administered at a dose of 200 mg, once every three weeks; wherein the anti-VEGFR-2 antibody is administered at a dose of 8 mg/kg on Day 1 and Day 8 of a three week cycle.

Another aspect of the invention is an anti-VEGFR-2 antibody comprising two light chains, each having the amino acid sequence of SEQ ID NO: 3 and two heavy chains, each having the amino acid sequence of SEQ ID NO: 4, for use in simultaneous, separate, or sequential combination with an anti-PD-1 antibody comprising two light chains, each having the amino acid sequence of SEQ ID NO: 5 and two heavy chains, each having the amino acid sequence of SEQ ID NO: 6, in the treatment of non-small cell lung cancer, locally advanced and unresectable or metastatic gastric or gastroesophageal junction adenocarcinoma, or urothelial cancer; wherein the anti-PD-1 antibody is administered at a dose of 200 mg, once every three weeks; wherein the anti-VEGFR-2 antibody is administered at a dose of 10 mg/kg once every three weeks.

Another aspect of the invention is an anti-VEGFR-2 antibody comprising two light chains, each having the amino acid sequence of SEQ ID NO: 3 and two heavy chains, each having the amino acid sequence of SEQ ID NO: 4, for use in simultaneous, separate, or sequential combination with an anti-PD-1 antibody comprising two light chains, each having the amino acid sequence of SEQ ID NO: 5 and two heavy chains, each having the amino acid sequence of SEQ ID NO: 6, in the treatment of non-small cell lung cancer; wherein the anti-PD-1 antibody is administered at a dose of 200 mg, once every three weeks; wherein the anti-VEGFR-2 antibody is administered at a dose of 10 mg/kg once every three weeks.

Another aspect of the invention is an anti-VEGFR-2 antibody comprising two light chains, each having the amino acid sequence of SEQ ID NO: 3 and two heavy chains, each having the amino acid sequence of SEQ ID NO: 4, for use in simultaneous, separate, or sequential combination with an anti-PD-1 antibody comprising two light chains, each having the amino acid sequence of SEQ ID NO: 5 and two heavy chains, each having the amino acid sequence of SEQ ID NO: 6, in the treatment of locally advanced and unresectable or metastatic gastric or gastroesophageal junction adenocarcinoma; wherein the anti-PD-1 antibody is administered at a dose of 200 mg, once every three weeks; wherein the anti-VEGFR-2 antibody is administered at a dose of 10 mg/kg once every three weeks.

Another aspect of the invention is an anti-VEGFR-2 antibody comprising two light chains, each having the amino acid sequence of SEQ ID NO: 3 and two heavy chains, each having the amino acid sequence of SEQ ID NO: 4, for use in simultaneous, separate, or sequential combination with an anti-PD-1 antibody comprising two light chains, each having the amino acid sequence of SEQ ID NO: 5 and two heavy chains, each having the amino acid sequence of SEQ ID NO: 6, in the treatment of urothelial cancer; wherein the anti-PD-1 antibody is administered at a dose of 200 mg, once every three weeks; wherein the anti-VEGFR-2 antibody is administered at a dose of 10 mg/kg once every three weeks.

Another aspect of the invention is a use of an anti-VEGFR-2 antibody comprising two light chains, each having the amino acid sequence of SEQ ID NO: 3 and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6, for the manufacture of a medicament for the treatment of non-small cell lung cancer, locally advanced and unresectable or metastatic gastric or gastroesophageal junction adenocarcinoma, urothelial cancer, or binary tract cancer; wherein the anti-PD-1 antibody is administered at a dose of 200 mg, once every three weeks.

Another aspect of the invention is a use of an anti-VEGFR-2 antibody comprising two light chains, each having the amino acid sequence of SEQ ID NO: 3 and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6, for the manufacture of a medicament for the treatment of locally advanced and unresectable or metastatic gastric or gastroesophageal junction adenocarcinoma or biliary tract cancer; wherein the anti-PD-1 antibody is administered at a dose of 200 mg, once every three weeks; wherein the anti-VEGFR-2 antibody is administered at a dose of 8 mg/kg on Day 1 and Day 8 of a three week cycle.

Another aspect of the invention is a use of an anti-VEGFR-2 antibody comprising two light chains, each having the amino acid sequence of SEQ ID NO: 3 and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6, for the manufacture of a medicament for the treatment of locally advanced and unresectable or metastatic gastric or gastroesophageal junction adenocarcinoma; wherein the anti-PD-1 antibody is administered at a dose of 200 mg, once every three weeks; wherein the anti-VEGFR-2 antibody is administered at a dose of 8 mg/kg on Day 1 and Day 8 of a three week cycle.

Another aspect of the invention is a use of an anti-VEGFR-2 antibody comprising two light chains, each having the amino acid sequence of SEQ ID NO: 3 and two heavy chains, each having the amino acid sequence of SEQ ID NO: 4, and an anti-PD-1 antibody comprising two light chains, each having the amino acid sequence of SEQ NO: 5 and two heavy chains, each having the amino acid sequence of SEQ ID NO: 6, for the manufacture of a medicament for the treatment of biliary tract cancer; wherein the anti-PD-1 antibody is administered at a dose of 200 mg, once every three weeks; wherein the anti-VEGFR-2 antibody is administered at a dose of 8 mg/kg on Day 1 and Day 8 of a three week cycle.

Another aspect of the invention is a use of an anti-VEGFR-2 antibody comprising two light chains, each having the amino acid sequence of SEQ ID NO: 3 and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6, for the manufacture of a medicament for the treatment of non-small cell lung cancer, locally advanced and unresectable or metastatic gastric or gastroesophageal junction adenocarcinoma, or urothelial cancer; wherein the anti-PD-1 antibody is administered at a dose of 200 mg, once every three weeks; wherein the anti-VEGFR-2 antibody is administered at a dose of 10 mg/kg once every three weeks.

Another aspect of the invention is a use of an anti-VEGFR-2 antibody comprising two light chains, each having the amino acid sequence of SEQ ID NO: 3 and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6, for the manufacture of a medicament for the treatment of non-small cell lung cancer; wherein the anti-PD-1 antibody is administered at a dose of 200 mg, once every three weeks; wherein the anti-VEGFR-2 antibody is administered at a dose of 10 mg/kg once every three weeks.

Another aspect of the invention is a use of an anti-VEGFR-2 antibody comprising two light chains, each having the amino acid sequence of SEQ ID NO: 3 and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6, for the manufacture of a medicament for the treatment of locally advanced and unresectable or metastatic gastric or gastroesophageal junction adenocarcinoma; wherein the anti-PD-1 antibody is administered at a dose of 200 mg, once every three weeks; wherein the anti-VEGFR-2 antibody is administered at a dose of 10 mg/kg once every three weeks.

Another aspect of the invention is a use of an anti-VEGFR-2 antibody comprising two light chains, each having the amino acid sequence of SEQ ID NO: 3 and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6, for the manufacture of a medicament for the treatment of urothelial cancer; wherein the anti-PD-1 antibody is administered at a dose of 200 mg, once every three weeks; wherein the anti-VEGFR-2 antibody is administered at a dose of 10 mg/kg once every three weeks.

Another aspect of the invention is a kit comprising an anti-VEGFR-2 antibody comprising two light chains, each having the amino acid sequence of SEQ ID NO: 3, and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6 for the treatment of non-small cell lung cancer, locally advanced and unresectable or metastatic gastric or gastroesophageal junction adenocarcinoma, urothelial cancer, or biliary tract cancer.

Another aspect of the invention is a combination comprising ramucirumab and pembrolizumab for simultaneous, separate or sequential use in the treatment of non-small cell lung cancer, locally advanced and unresectable or metastatic gastric or gastroesophageal junction adenocarcinoma, urothelial cancer, or biliary tract cancer; wherein pembrolizumab is administered at a dose of 200 mg, once every three weeks. Another aspect of the invention is a combination comprising ramucirumab and pembrolizumab for simultaneous, separate or sequential use in the treatment of advanced gastric or gastroesophageal junction adenocarcinoma; wherein pembrolizumab is administered at a dose of 200 mg, once every three weeks. Another aspect of the invention is ramucirumab for use in simultaneous, separate or sequential treatment with pembrolizumab in the treatment of non-small cell lung cancer, locally advanced and unresectable or metastatic gastric or gastroesophageal junction adenocarcinoma, urothelial cancer, or biliary tract cancer; wherein pembrolizumab is administered at a dose of 200 mg, once every three weeks. Another aspect of the invention is ramucirumab for use in simultaneous, separate or sequential treatment with pembrolizumab in the treatment of advanced gastric or gastroesophageal junction adenocarcinoma; wherein pembrolizumab is administered at a dose of 200 mg, once every three weeks. Another aspect of the invention is pembrolizumab and ramucirumab for use in the treatment of a non-small cell lung cancer, locally advanced and unresectable or metastatic gastric or gastroesophageal junction adenocarcinoma, urothelial cancer, or biliary tract cancer in a patient, wherein the patient has PD-L1 negative or positive status; wherein pembrolizumab is administered at a dose of 200 mg, once every three weeks. Another aspect of the invention is pembrolizumab and ramucirumab for use in the treatment of an advanced gastric or gastroesophageal junction adenocarcinoma in a patient, wherein the patient has PD-L1 negative or positive status; wherein pembrolizumab is administered at a dose of 200 mg, once every three weeks. Another aspect of the invention is use of ramucirumab and pembrolizumab in the manufacture of a medicament for the treatment of a patient with non-small cell lung cancer, locally advanced and unresectable or metastatic gastric or gastroesophageal junction adenocarcinoma, urothelial cancer, or biliary tract cancer; wherein pembrolizumab is administered at a dose of 200 mg, once every three weeks; optionally, wherein the patient has PD-L1 negative status. Another aspect of the invention is use of ramucirumab and pembrolizumab in the manufacture of a medicament for the treatment of a patient with advanced gastric or gastroesophageal junction adenocarcinoma; wherein pembrolizumab is administered at a dose of 200 mg, once every three weeks; optionally, wherein the patient has PD-1 negative status. Another aspect of the invention is a method of treating non-small cell lung cancer, locally advanced and unresectable or metastatic gastric or gastroesophageal junction adenocarcinoma, urothelial cancer, or biliary tract cancer in a patient, comprising administering an effective amount of ramucirumab in combination with pembrolizumab to the patient in need thereof, provided that the patient is selected for treatment if the patient has PD-L1 negative or positive status; wherein pembrolizumab is administered at a dose of 200 mg, once every three weeks. Another aspect of the invention is a method of treating advanced gastric or gastroesophageal junction adenocarcinoma in a patient, comprising administering an effective amount of ramucirumab in combination with pembrolizumab to the patient in need thereof, provided that the patient is selected for treatment if the patient has PD-L1 negative or positive status; wherein pembrolizumab is administered at a dose of 200 mg, once every three weeks. Another aspect of the invention is a method of treating non-small cell lung cancer, locally advanced and unresectable or metastatic gastric or gastroesophageal junction adenocarcinoma, urothelial cancer, or biliary tract cancer in a patient, comprising testing the patient for the presence of PD-L1 prior to administering ramucirumab in combination with pembrolizumab, and administering to the patient an effective amount of ramucirumab in combination with pembrolizumab if the patient has positive or negative PD-L1 status; wherein pembrolizumab is administered at a dose of 200 mg, once every three weeks. Another aspect of the invention is a method of treating advanced gastric or gastroesophageal junction adenocarcinoma in a patient, comprising testing the patient for the presence of PD-L1 prior to administering ramucirumab in combination with pembrolizumab, and administering to the patient an effective amount of ramucirumab in combination with pembrolizumab if the patient has positive or negative PD-L1 status; wherein pembrolizumab is administered at a dose of 200 mg, once every three weeks.

This disclosure provides a kit comprising an anti-VEGFR-2 antibody comprising two light chains, each having the amino acid sequence of SEQ ID NO: 3, and two heavy chains, each having the amino acid sequence of SEQ ID NO: 4, and an anti-PD-1 antibody comprising two light chains, each having the amino acid sequence of SEQ ID NO: 5 and two heavy chains, each having the amino acid sequence of SEQ ID NO: 6 for the treatment of non-small cell lung cancer, locally advanced and unresectable or metastatic gastric or gastroesophageal junction adenocarcinoma, urothelial cancer, or biliary tract cancer.

This disclosure provides a combination comprising ramucirumab and pembrolizumab for simultaneous, separate or sequential use in the treatment of non-small cell lung cancer, locally advanced and unresectable or metastatic gastric or gastroesophageal junction adenocarcinoma, urothelial cancer, or biliary tract cancer; wherein pembrolizumab is administered at a dose of 200 mg, once every three weeks.

This disclosure provides a combination comprising ramucirumab and pembrolizumab for simultaneous, separate or sequential use in the treatment of advanced gastric or gastroesophageal junction adenocarcinoma; wherein pembrolizumab is administered at a dose of 200 mg, once every three weeks.

This disclosure provides ramucirumab for use in simultaneous, separate or sequential treatment with pembrolizumab in the treatment of non-small cell lung cancer, locally advanced and unresectable or metastatic gastric or gastroesophageal junction adenocarcinoma, urothelial cancer, or biliary tract cancer; wherein pembrolizumab is administered at a dose of 200 mg, once every three weeks.

This disclosure provides ramucirumab for use in simultaneous, separate or sequential treatment with pembrolizumab in the treatment of advanced gastric or gastroesophageal junction adenocarcinoma; wherein pembrolizumab is administered at a dose of 200 mg, once every three weeks.

This disclosure provides pembrolizumab and ramucirumab for use in the treatment of a non-small cell lung cancer, locally advanced and unresectable or metastatic gastric or gastroesophageal junction adenocarcinoma, urothelial cancer, or biliary tract cancer in a patient, wherein the patient has PD-L1 negative or positive status; wherein pembrolizumab is administered at a dose of 200 mg, once every three weeks.

This disclosure provides pembrolizumab and ramucirumab for use in the treatment of an advanced gastric or gastroesophageal junction adenocarcinoma in a patient, wherein the patient has PD-L1 negative or positive status; wherein pembrolizumab is administered at a dose of 200 mg, once every three weeks.

This disclosure provides the use of ramucirumab and pembrolizumab in the manufacture of a medicament for the treatment of a patient with non-small cell lung cancer, locally advanced and unresectable or metastatic gastric or gastroesophageal junction adenocarcinoma, urothelial cancer, or biliary tract cancer; wherein pembrolizumab is administered at a dose of 200 mg, once every three weeks.

This disclosure provides the use of ramucirumab and pembrolizumab in the manufacture of a medicament for the treatment of a patient with advanced gastric or gastroesophageal junction adenocarcinoma; wherein pembrolizumab is administered at a dose of 200 mg, once every three weeks.

This disclosure provides a combination comprising ramucirumab and pembrolizumab for simultaneous, separate or sequential use in the treatment of advanced gastric or gastroesophageal junction adenocarcinoma; wherein pembrolizumab is administered at a dose of 200 mg, once every three weeks; wherein the patient has PD-L1 negative status.

This disclosure provides ramucirumab for use in simultaneous, separate or sequential treatment with pembrolizumab in the treatment of non-small cell lung cancer, locally advanced and unresectable or metastatic gastric or gastroesophageal junction adenocarcinoma, urothelial cancer, or biliary tract cancer; wherein pembrolizumab is administered at a dose of 200 mg, once every three weeks; wherein the patient has PD-L1 negative status.

This disclosure provides a method of treating non-small cell lung cancer, locally advanced and unresectable or metastatic gastric or gastroesophageal junction adenocarcinoma, urothelial cancer, or biliary tract cancer in a patient, comprising administering an effective amount of ramucirumab in combination with pembrolizumab to the patient in need thereof, provided that the patient is selected for treatment if the patient has PD-L1 negative or positive status; wherein pembrolizumab is administered at a dose of 200 mg, once every three weeks.

This disclosure provides a method of treating advanced gastric or gastroesophageal junction adenocarcinoma in a patient, comprising administering an effective amount of ramucirumab in combination with pembrolizumab to the patient in need thereof, provided that the patient is selected for treatment if the patient has PD-L1 negative or positive status; wherein pembrolizumab is administered at a dose of 200 mg, once every three weeks.

This disclosure provides a method of treating non-small cell lung cancer, locally advanced and unresectable or metastatic gastric or gastroesophageal junction adenocarcinoma, urothelial cancer, or biliary tract cancer in a patient, comprising testing the patient for the presence of PD-L1 prior to administering ramucirumab in combination with pembrolizumab, and administering to the patient an effective amount of ramucirumab in combination with pembrolizumab if the patient has positive or negative PD-L1 status; wherein pembrolizumab is administered at a dose of 200 mg, once every three weeks.

This disclosure provides a method of treating advanced gastric or gastroesophageal junction adenocarcinoma in a patient, comprising testing the patient for the presence of PD-L1 prior to administering ramucirumab in combination with pembrolizumab, and administering to the patient an effective amount of ramucirumab in combination with pembrolizumab if the patient has positive or negative PD-L1 status; wherein pembrolizumab is administered at a dose of 200 mg, once every three weeks.

As used herein, the term “VEGFR-2” refers to Vascular Endothelial Growth Factor Receptor 2, which is known in the art. VEGFR-2 is also known as KDR. A non-limiting example of ramucirumab is CYRAMZA® with CAS registry number 947687-13-0. Ramucirumab is an anti-VEGFR-2 Ab comprising two light chains, each of whose amino acid sequence is that given in SEQ ID NO: 3, and two heavy chains, each of whose amino acid sequence is that given in SEQ ID NO: 4. The light chain variable region of ramucirumab is that given in SEQ ID NO: 1. The heavy chain variable region of ramucirumab is that given in SEQ ID NO: 2. The antibody selected will have a sufficiently strong binding affinity for VEGFR-2. For example, the antibody will generally bind VEGFR-2 with a K_{d} value of between about 100 nM-about 1 pM. Antibody affinities may be determined by a surface plasmon resonance based assay (such as the BIAcore assay is described in WO2005/012359); enzyme-linked immunosorbent assay (ELISA); and competition assays (e.g. a radiolabeled antigen binding assay (RIA)), for example. In one embodiment, Kd is measured by a RIA performed with ramucirumab.

As used herein, the term “PD-1” refers to human PD-1 which is known in the art. A non-limiting example of pembrolizumab is KEYTRUDA®. Pembrolizumab is an anti-PD-1 antibody comprising two light chains, each of whose amino acid sequence is that given in SEQ ID NO: 5, and two heavy chains, each of whose amino acid sequence is that given in SEQ ID NO: 6. The light chain variable region of ramucirumab is that given in SEQ ID NO: 7. The heavy chain variable region of ramucirumab is that given in SEQ ID NO: 8.

Unless indicated otherwise, the term “antibody” refers to an immunoglobulin molecule comprising two heavy chains (HC) and two light chains (LC) interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “light chain variable region” or “LCVR” refers to a portion of a light chain of an antibody molecule that includes amino acid sequences of CDRs and framework regions FRs.

As used herein, the term “heavy chain variable region” “HCVR” refers to a portion of a heavy chain of an antibody molecule that includes amino acid sequences of CDRs and FRs.

As used herein, the term “kit” refers to a package comprising at least two separate containers, wherein a first container contains ramucirumab, and a second container contains pembrolizumab. A “kit” may also include instructions to administer all or a portion of the contents of these first and second containers to a cancer patient, preferably a non-small cell lung cancer patient.

As used herein, the terms “treating,” “treat,” or “treatment” refer to restraining, slowing, lessening, reducing, or reversing the progression or severity of an existing symptom, disorder, condition, or disease, or ameliorating clinical symptoms of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Treatment can also mean prolonging survival as compared to expected survival if not receiving treatment. Those in need of treatment include those already with the disease. In one embodiment, the present invention can be used as a medicament.

As used herein, the term “patient” refers to a mammal, preferably a human.

As used herein, the term “cancer” refers to or describe the physiological condition in patients that is typically characterized by unregulated cell proliferation. Included in this definition are benign and malignant cancers.

As used herein, the term “effective amount” refers to the amount or dose of ramucirumab and pembrolizumab which provides an effective response in the patient under diagnosis or treatment.

As used herein, the term “effective response” of a patient or a patient's “responsiveness” to treatment with a combination of agents refers to the clinical or therapeutic benefit imparted to a patient upon administration of ramucirumab and pembrolizumab.

Generally, dosage regimens may be adjusted to provide the optimum desired response (e.g., a therapeutic response). Treatment dosages may be titrated using routine methods known to those of skill in the art to optimize safety and efficacy. Dosing schedules will typically range from a single bolus dosage or continuous infusion, to multiple administrations per day (e.g., every 4-6 hours), or as indicated by the treating physician and the patient's condition. Dosing frequencies of the antibody will be determined by the physicians treating the patient and may be given daily, three times per week, weekly, every two weeks, or less often, and more preferably every three weeks. Dosing amounts of the antibodies will also be determined by the physicians treating the patient and may fall within customary ranges.

In some instances, dosage levels below the lower limit of the aforesaid dosing for the antibodies of the invention may be more than adequate, while in other cases larger doses may be employed with acceptable side effects, and therefore the above dosage amount is not intended to limit the scope of the invention in any way.

Ramucirumab may be administered from 2 to 20 mg/kg, weekly, every two weeks, or every three weeks, depending on tumor type, and patient factors. Preferably, ramucirumab may be administered at 10 mg/kg intravenously on day 1 of a 21-day cycle.

Pembrolizumab may be administered from 1 mg/kg to 10 mg/kg, every two weeks. In one embodiment, pembrolizumab is administered at a dose of 1, 2, 3, 5 or 10 mg/kg at intervals of about 14 days (±2 days) or about 21 days (±2 days) or about 30 days (±2 days) throughout the course of treatment. In another embodiment, about 200 mg of pembrolizumab is administered as an intravenous infusion over 25 to 40 minutes, preferably 30 minutes, every 3 weeks.

The route of administration may be varied in any way, limited by the physical properties of the drugs and the convenience of the patient and the caregiver. Preferably, ramucirumab and pembrolizumab are formulated for parenteral administration, such as intravenous or subcutaneous administration.

As used herein, the phrase “in combination with” refers to the administration of ramucirumab and pembrolizumab.

The therapeutically effective amount of the treatment of the invention can be measured by various endpoints commonly used in evaluating cancer treatments, including, but not limited to: extending survival (including OS and PFS) resulting in an objective response (including a CR or a PR); tumor regression, tumor weight or size shrinkage, longer time to disease progression, increased duration of survival, longer PFS, improved OS rate, increased duration of response, and improved quality of life and/or improving signs or symptoms of cancer.

As used herein, the term “progressive disease” (PD) refers to least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

As used herein, the term “partial response,” (PR) refers to at least a 30% decrease in the sum of diameters of target lesions, taking as reference the baseline sum diameters.

As used herein, the term “complete response” (CR) refers to the disappearance of all target lesions with the short axes of any target lymph nodes reduced to <10 mm.

As used herein, the term “stable disease” (SD) refers to neither sufficient shrinkage to qualify for PR nor sufficient increase to qualify for PD, taking as reference the smallest sum of diameters while on study.

As used herein, the term “not evaluable” (NE) refers to when an incomplete radiologic assessment of target lesions is performed or there is a change in the method of measurement from baseline that impacts the ability to make a reliable evaluation of response.

As used herein, the term “objective response rate” (ORR) is equal to the proportion of patients achieving a best overall response of partial or complete response (PR+CR) according to RECIST 1.1.

As used herein, the term “overall survival” (OS) refers to the percentage of patients remaining alive for a defined period of time, such as 1 year, 5 years, etc. from the time of diagnosis or treatment. In a preferred embodiment, OS refers to the time from the date of randomization in the Study to the date of death from any cause. If the patient is alive at the end of the follow-up period or is lost to follow-up, OS data is censored on the last date the patient is known to be alive. Overall survival is evaluated by the Kaplan-Meier method, and a 95% confidence interval (CI) is provided for the median OS in each treatment arm.

As used herein, the term “progression-free survival” (PFS) refers to the patient remaining alive without the cancer progressing or getting worse. In a preferred aspect of the invention, PFS is defined as the time from randomization in the Study until the first radiographic documentation of objective progression as defined by RECIST (Version 1.1), or death from any cause. Patients who die without a reported prior progression will be considered to have progressed on the day of their death. Patients who did not progress or are lost to follow-up will be censored at the day of their last radiographic tumor assessment.

As used herein, the term “disease control rate” (DCR) refers to lack of disease progression and rate thereof. It refers to the group of patients with a best overall response categorized as CR, PR or SD (specifically excluding the patients with PD), wherein the best overall response is the best response recorded from the start of treatment until PD.

As used herein, the term “clinical benefit rate,” refers to SD or better at 12 weeks. The tumor response rate of SD or better (i.e. CR+PR+SD) at 12 weeks is defined as the proportion of patients with a response of SD or better, as defined by RECIST 1.1, at 12 weeks following the first dose of study therapy. Patients will be considered “failure” if they die or if radiographic evaluation indicates a response of PD at 12 weeks or before.

As used herein, the term “extending survival” is meant as increasing OS or PFS in a treated patient relative to i) an untreated patient, ii) a patient treated with less than all of the anti-tumor agents in a particular combination therapy, or iii) a control treatment protocol. Survival is monitored following the initiation of treatment or following the initial diagnosis of cancer.

As used herein, the term “best overall response” is the best response recorded from the start of the study treatment until the earliest of objective progression or start of new anticancer therapy, taking into account any requirement for confirmation. The patient's best overall response assignment will depend on the findings of both target and nontarget disease and will also take into consideration the appearance of new lesions. The best overall response will be calculated via an algorithm using the assessment responses provided by the investigator over the course of the trial.

The following examples illustrate the unexpected improvement of the combination of ramucirumab and pembrolizumab in certain cancers.

### An Open-Label, Multicenter, Phase 1 Study of Ramucirumab Plus Pembrolizumab in Patients with Locally Advanced and Unresectable or Metastatic Gastric or Gastroesophageal Junction Adenocarinoma, Non-Small Cell Lung Cancer, Transitional Cell Carcinoma of the Urothelium, or Biliary Tract Cancer

### Mar. 14, 2016 Data Cut Off

Study I4T-MC-JVDF is an open-label, multicenter Phase 1 study to evaluate the safety and efficacy of ramucirumab in combination with pembrolizumab, Phase 1a (dose-limiting toxicity (DLT)) and Expansion Phase 1b (safety and preliminary efficacy) includes patients with locally advanced and unresectable or metastatic gastric or gastroesophageal junction adenocarcinoma; non-small cell lung cancer (NSCLC); transitional cell carcinoma of the urothelium (urothelial cancer); or biliary tract cancer (BTC). If sufficient tolerability and preliminary efficacy are demonstrated in Phase 1, the protocol will be amended to further evaluate efficacy and safety, and re-submitted accordingly.

The primary objectives of Phase Ia and Ib of the Study are to assess the safety and tolerability of two dosing regimens of ramucirumab plus pembrolizumab. The primary endpoints of Phase Ia and Ib of the Study are dose-limiting toxicities, observed during a 21-day treatment cycle, and safety (include but not limited to): TEAEs, SAEs, deaths, laboratory abnormalities, vital signs, and physical exams. The secondary objectives of Phase Ia and Ib of the Study are to characterize the pharmacokinetics of ramucirumab when coadministered with pembrolizumab. The secondary endpoints of Phase Ia and Ib of the Study are pharmacokinetics (PK): Cmin (minimum concentration) and approximate Cmax (maximum concentration) of ramucirumab in serum.

The secondary objectives of Phase Ib of the Study are to assess the preliminary efficacy of ramucirumab plus pembrolizumab. The secondary endpoints of Phase Ib of the Study are ORR (RECIST 1.1 and irRECIST) and DCR, duration of response (DOR), time to response (TTR), PFS, and OS.

The tertiary objectives of Phase Ib of the Study are to explore the association between biomarkers and clinical outcomes, to characterize biomarker measures of immune functioning and angiogenesis, and to assess immunogenicity of ramucirumab when co-administered with pembrolizumab. The tertiary endpoints of Phase Ib of the Study are biomarker research on genetic and circulating factors, and immunogenicity of anti-ramucirumab antibody.

In Dose Limiting Toxicity (DLT) Phase 1a, patients are treated for up to 21 days (1 cycle), and patients without a DLT may continue in Expansion Phase 1b. Patients are administered ramucirumab 8 mg/kg on Day 1 and Day 8 and pembrolizumab 200 mg (fixed dose) on Day 1, in 3 patients with gastric-gastroesophageal (GEJ) cancer or biliary tract cancer (BTC). Patients are administered ramucirumab 10 mg/kg and pembrolizumab 200 mg (fixed dose) on Day 1, in 3 patients with either gastric-GEJ, NSCLC, or urothelial cancer. Up to 12 DLT-evaluable patients (up to 6 enrolled in each dosing schedule) are treated.

In Expansion Phase Ib, the duration continues until approximately 2 years after the first patient received study treatment. Individual patients may continue treatment for up to 35 cycles (approximately 2 years), until confirmed progressive disease or discontinuation for any other reason. For Treatment Cohorts, Schedule 1 Dose: Gastric-GEJ (2nd-3rd Line) Cohort A (15 patients), BTC (2nd-3rd Line) Cohort A1 (25 patients), and Gastric-GEJ (1st Line) Cohort A2 (25 patients). In Schedule 2 Dose: Gastric-GEJ (2nd-3rd Line) Cohort B (15 patients), NSCLC (2nd-4th Line) Cohort C (25 patients), Urothelial (2nd-4th Line) Cohort D (25 patients), and NSCLC (1st Line) Cohort E (25 patients). Total approximately 155 patients.

For first line NSCLC, patient receive study treatment after PD-L1 expression has been confirmed to be at least 1%.

This invention discloses Phase Ia results for 27 patients in Cohort C (NSCLC) of the Study. 20 patients (74.0%) remain on Study. 7 patients (26.0%) are no longer on Study due to either progressive disease (5 patients, 19.0%), death (1 patient, 4%), or adverse event (1 patient, 4.0%). The median duration of therapy was 18 weeks, the median number of cycles was 6, and 24 patients completed greater than or equal to 3 cycles. No unexpected safety events were reported and grade ¾ toxicities were low (9.0%) in patients with NSCLC, gastric/GEJ cancer or UC.

7 of the 27 patients (26.0%) in Cohort C demonstrated ORR, and 23 of the 27 patients (85.0%) in Cohort C demonstrated DCR. 1 patient had complete response (CR) (4.0%), 6 patients had partial response (PR) (22.0%); 16 patients had stable disease (SD) (59.0%); 3 patients had progressive disease (PD) (11.0%); and one patient was not evaluable (4.0%). Surprisingly, 85.0% of patients experienced a decrease in target lesions (PR, CR or SD).

Additionally, patients were analyzed for PD-L1 status. PD-L1 expression was assessed using PD-L1 22C3 IHC pharmDx assay (Dako). PD-L1 status was classified using tumor proportion score as strong positive (≥50%), weak positive (1-49%), or negative in NSCLC; positive (≥1%) or negative only for gastric/GEJ and UC.

Of the 27 patients in Cohort C, 6 patients had strong positive PD-L1 status; 3 patients had weak positive PD-L1 status (for a total of 9 positive); 10 patients had negative PD-L1 status; and 8 patients had unknown PD-L1 status.

Preliminary activity was observed in 7 patients with PD-L1 negative status or PD-L1 positive status. 4 of 6 strong positive PD-L1 status patients responded (all PRs); and 1 of 8 unknown PD-L1 status responded (PR). Surprisingly, 2 of 10 negative PD-L1 status patients responded (1 patient with a complete response, and one patient with a partial response).

Median time to response was 1.45 months and median duration of response has not been readied. Median PFS has not been reached (95% CI, 3.98 to NR). Median duration of treatment is 6.8+ months.

### Jun. 23, 2016 Data Cut Off

8 of the 27 patients (30.0%) in Cohort C demonstrated an ORR, and 23 of the 27 patients (85%) in Cohort C demonstrated DCR. 1 patient had complete response (CR) (3.7%), 7 patients had partial response (PR) (25.9%); 15 patients had stable disease (SD) (55.6%); 3 patients had progressive disease (PD) (11.1%); and one patient was not evaluable (3.7%).

Of the 27 patients in Cohort C, 7 patients had strong positive PD-L1 status; 4 patients had weak positive PD-L1 status (for a total of 11 positive); 10 patients had negative PD-L1 status; and 6 patients had unknown PD-L1 status.

Preliminary activity (ORR) was observed in 8 patients with PD-L1 negative status or PD-L1 positive status. 5 of 7 strong positive PD-L1 status patients responded (all PRs); and 1 of 6 unknown PD-L1 status responded (PR). Surprisingly, 2 of 10 negative PD-L1 status patients responded (1 patient with a complete response, and 1 patient with a partial response). At the time of the data cut, tumor responses were still ongoing in all of the 8 responders, including the patient with complete response. Median Progression Free Survival was not reached in Cohort C. Among the 8 responders, PFS was all censored (no disease progression or death events), and it ranged from 5.3+ to 6.9+ months.

40 Gastric and GEJ cancer patients are currently enrolled in Cohorts A and Cohorts B (Cohort A: n=23; Cohort B: n=17). Of the 40 patients, 60% of the patients have GEJ and 37.5% of the patients have gastric cancer, with 48% of the 40 patients having a PD-L1 positive status. As of the data cut-off, the median duration of treatment was 2.1 months and 4.1 months for Cohort A and B, respectively. Three (7.5%) patients (PD-L1 negative n=1; PD-L1 positive n=2) have responded (1 patient confirmed and 2 patients unconfirmed PR) to treatment with a 45% disease control rate. Median PFS was 2.10 months (95% CI, 1.18 to 4.04) and 2.60 months (1.38,—not reached) for Cohorts A and B respectively. Fifteen (37.5%) patients, including all responders, were on treatment at the data cutoff.

As of 21 Oct. 2016, 27 patients with previously treated advanced NSCLC received ramucirumab at 10 mg/kg on Day 1 with pembrolizumab at 200 mg on Day 1 q3W. The median age was 65, 78% were male, 96% had a history of smoking, 78% had adenocarcinoma and 15% had squamous-cell carcinoma. Sixteen (59%) patients received ≥2 and 4 (15%) patients received ≥3 prior treatment regimens for their disease. Treatment related adverse events (TRAEs) occurred in 24 (89%) patients, most commonly hypertension (22%) and asthenia (18.5%). Three (11%) patients experienced grade 3 TRAEs (adrenal insufficiency, hyponatremia, delirium, hypertension, and infusion related reaction). No grade 4-5 TRAEs occurred. Median PFS was 8.8 mo (95% CI 4.6 to 11.3) and the estimated rate of overall survival at 6 months was 80.2%. The ORR was 30% with a median time to response of 1.4 months. The duration of response has not been reached and responses occurred in all PD-L1 groups and both histological subtypes. The disease control rate was 85%. Twelve (44%) patients are still on study treatment, including all responders. Ramucirumab in combination with pembrolizumab demonstrated antitumor activity in all PD-L1 groups and both histological subtypes. The safety profile was consistent with monotherapy treatment for each drug, with no additive toxicities. The study was amended to include patients with first-line advanced NSCLC; enrollment is ongoing.

As of 21 Oct. 2016, TABLE A shows the results from the study of ramucirumab in combination with pembrolizumab in patients with advanced gastric or gastroesophageal junction adenocarcinoma.

TABLE A

| | Cohort A (n = 24) | Cohort B (n = 17) | Total 9 (n = 41) |
|---|---|---|---|
| Objective | 3 | 1 | 4 (3 confirmed |
| Response Rate | | | and 1 |
| | | | unconfirmed |
| | | | response); 10% |
| | | | (12% in response |
| | | | evaluable |
| | | | population) |
| Disease Control | 10 | 9 | 19 |
| Rate (in patients | | | |
| with best response | | | |
| of CR, PR, or SD) | | | |
| Median Duration | NR | NR | NA |
| of Response | | | |
| (months) | | | |
| Median Time to | 1.4 (1.4, 2.7) | 4.1 (—, —) | NA |
| Response, months | | | |
| (95% CI) | | | |
| Duration of Stable | 4.6 (2.2, —) | NR (2.6, —) | NA |
| Disease | | | |
| Complete | — | — | — |
| Response (CR) | | | |
| Partial Response | 3 | 1 | 4* |
| (PR) | | | |
| Stable Disease | 7 | 8 | 15 |
| (SD) | | | |
| Progressive | 9 | 6 | 15 |
| Disease (PD) | | | |
| Not Evaluable | 5 | 2 | 7 |

NR = not reached;
NA = not applicable;
* = 2/4 partial responders are PD-L1 status negative, 1/4 partial responder is PD-L1 status positive, and 1/4 partial responder is unknown.

As of 21 Nov. 2016, 24 patients have been treated in Cohort D. The median age was 63 years, 58% were male, 50% had ECOG PS 0, 50% were PD-L1 positive and 63% received study treatment as third or subsequent line. Median duration of treatment was 2.14 mo and 2.37 mo for ramucirumab and pembrolizumab, respectively. Antitumor activity was seen in heavily pretreated patients with urothelial carcinoma and in those who were PD-L1 positive (n=3 responders). Disease control rate was 50%. Median progression-free and overall survival were 1.9 mo (95% CI 1.2-2.8) and 6.4 mo (95% CI 2.5-NR), respectively. Median duration of response has not been reached. Four patients remain on treatment.

TABLE B

| | Cohort D (n = 24) | |
|---|---|---|
| Objective response rate | 3 | (13)^{a} |
| Disease control rate^{b} | 12 | (50) |
| Median duration of response, mo (95% CI) | NR | (4.6-NR) |
| Median time to response, | 2.8 | (1.3-5.5) |
| mo (95% CI) | | |
| Duration of Stable disease | 2.8 | (1.9-NR) |
| Complete response (CR) | — | |
| Partial response (PR) | 3 | (13) |
| Stable disease (SD) | 9 | (38) |
| Progressive disease (PD) | 11 | (46) |
| Not Evaluable | 1 | (4) |

^{a}All responders were PD-L1 positive;
^{b}Patients with best response of CR, PR, or SD;
NR = not reached

As of 21 Nov. 2016, 26 patients with biliary tract cancer were enrolled. Median age was 63 years, 69% were female, 54% had ECOG PS of 1, 38% received study treatment as third or subsequent line and PD-L1 status is pending. The median duration of therapy was 2 months. Overall, 22 (85%) patients experienced a treatment-related AE (TRAE), most commonly hypertension (31%), fatigue (23%) and nausea (23%). Grade 3-4 TRAEs occurred in nine (35%) patients (hypertension [n=5], diarrhea, duodenal ulcer, hematemesis, neutropenia and transaminases increased). No treatment-related deaths occurred. One (4%) patient discontinued treatment due to an adverse event (transaminases increased). One (4%) patient had partial response (unconfirmed), 8 (31%) patients had stable disease, and 12 (46%) patients had progressive disease as their best response. Five (19%) patients were not evaluable for response at the time of analysis. Median progression-free survival was 1.5 months (95% CI 1.4 to 2.8) and median overall survival has not been reached. Nine (35%) patients remain on treatment.

SEQUENCE LISTING

SEQ ID NO: 1
DIQMTQSPSSVSASIGDRVTITCRASQGIDNWLGWYQQKPGKAPIKLLIY
DASNLDTGVPSRFSGSGSGTYFTLTISSLQAEDFAVYFCQQAKAFPPTFG
GGTKVDIK
SEQ ID NO: 2
EVQLVQSGGGLVKPGGSLRLSCAASGFTFSSYSMNWVRQAPGKGLEWVSS
ISSSSSYIYYADSVKGRFTISRDNAKNSLYLQMNSLRAFDTAVYYCARVT
DAFDIWGQGTMVTVSS
SEQ ID NO: 3
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIKRTVAAPSVFIFPPSDEQLKSGTASVVCLLNNFYPREAKVQWKV
DNALQSGNSQESVTEQDSKDSFYSLSSTLTLSKADYEKHKVYACEVTHQG
LSSPVTKSFNRGEC
SEQ ID NO: 4
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSSASTKGPSVLPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPFVKFNWYVDGVEVHNAKTKPREEQYNSTYR
VVSVLTVLHQDWLNGKEYKCKVSNKALPAPIEKTISKARGQPREPQVYTL
PPSREEMTKNQVSLTCLVKGFYPSDIAVEWESNGQPENNYKTTPPVLDSD
GSFFLYSKLTVDKSRWQQGNVFSCSVMHEALIINHYTQKSLSLSPGK
SEQ ID NO: 5
EIVLTQSPAT LSLSPGERAT LSCRASKGVS TSGYSYLHWY
QQKPGQAPRL LIYLASYLES GVPARFSGSG SGTDFTLTIS
SLEPEDFAVY YCQHSRDLPL TFGGGTKVEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVF KSFNRGEC
SEQ ID NO: 6
QVQLVQSGVE VKKPGASVKV SCKASGYTFT NYYMYWVRQA
PGQGLEWMGG INPSNGGTNF NEKFKNRVTL TTDSSTTTAY
MELKSLQFDD TAVYYCARRD YRFDMGFDYW GQGTTVTVSS
ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRVES KYGPPCPPCP APEFLGGPSV
FLFPPKPKDT LMISRTPEVT CVVVDVSQED PEVQFNWYND
GVEVHNAKTK PREEQFNSTY RVVSVLTVLH QDWLNGKEYK
CKVSNKGLPS SIEKTISKAK GQPREPQVYT LPPSQEEMTK
NQVSLTCLVK GFYPSDIAVE WESNGQPENN YKTTPPVLDS
DGSFFLYSRL TVDKSRWQEG NVFSCSVMHE ALHNHYTQKS
LSLSLGK
SEQ ID NO: 7
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIK
SEQ ID NO: 8
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSS

1. A method of treating non-small cell lung cancer, locally advanced and unresectable or metastatic gastric or gastroesophageal junction adenocarcinoma, urothelial cancer, or biliary tract cancer in a patient, comprising administering to the patient in need of such treatment an effective amount of an anti-VEGFR-2 antibody comprising two light chains, each having the amino acid sequence of SEQ ID NO: 3 and two heavy chains, each having the amino acid sequence of SEQ ID NO: 4, and an effective amount of an anti-PD-1 antibody comprising two light chains, each having the amino acid sequence of SEQ ID NO: 5 and two heavy chains, each having the amino acid sequence of SEQ ID NO: 6; wherein the anti-PD-1 antibody is administered at a dose of 200 mg, once every three weeks.

2. The method of claim 1, wherein locally advanced and unresectable or metastatic gastric or gastroesophageal junction adenocarcinoma or biliary tract cancer is treated and the anti-VEGFR-2 antibody is administered at a dose of 8 mg/kg on Day 1 and Day 8 of a three week cycle.

3. The method of claim 1, wherein locally advanced and unresectable or metastatic gastric or gastroesophageal junction adenocarcinoma is treated and the anti-VEGFR-2 antibody is administered at a dose of 8 mg/kg on Day 1 and Day 8 of a three week cycle.

4. The method of claim 1, wherein biliary tract cancer is treated, and the anti-VEGFR-2 antibody is administered at a dose of 8 mg/kg on Day 1 and Day 8 of a three week cycle.

5. The method of claim 1, wherein non-small cell lung cancer, locally advanced and unresectable or metastatic gastric or gastroesophageal junction adenocarcinoma, or urothelial cancer is treated, and the anti-VEGFR-2 antibody is administered at a dose of 10 mg/kg once every three weeks.

6. The method of claim 1, wherein non-small cell lung cancer is treated, and the anti-VEGFR-2 antibody is administered at a dose of 10 mg/kg once every three weeks.

7. The method of claim 1, wherein locally advanced and unresectable or metastatic gastric or gastroesophageal junction adenocarcinoma is treated, and the anti-VEGFR-2 antibody is administered at a dose of 10 mg/kg once every three weeks.

8. The method of claim 1, wherein urothelial cancer is treated, and the anti-VEGFR-2 antibody is administered at a dose of 10 mg/kg once every three weeks.

9-24. (canceled)


SEQUENCE LISTING (8 sequences)

SEQ ID NO: 1 - protein, 107 aa, Artificial Sequence [sequence omitted]

SEQ ID NO: 2 - protein, 116 aa, Artificial Sequence [sequence omitted]

SEQ ID NO: 3 - protein, 214 aa, Artificial Sequence
  Synthetic Construct
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 446 aa, Artificial Sequence
  Synthetic Construct
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 447 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 111 aa, Artificial Sequence [sequence omitted]

SEQ ID NO: 8 - protein, 120 aa, Artificial Sequence [sequence omitted]